(19) United States
(12) Patent Application Publication
(10) Pub. No.: US 2010/0055666 A1
(43) Pub. Date: Mar. 4, 2010

(54) BIOSENSOR WITH EVANESCENT WAVEGUIDE AND INTEGRATED SENSOR

(75) Inventors: Reinhold Wimberger-Friedl, Eindhoven (NL); Nigel David Young, Redhill (GB); Pieter Jan Van Der Zaat, Eindhoven (NL)

Correspondence Address: PHILIPS INTELLECTUAL PROPERTY & STANDARDS, P.O. BOX 3001, BRIARCLIFF MANOR, NY, 10510, US

(73) Assignee: KONINKLIJKE PHILIPS ELECTRONICS N.V., EINDHOVEN (NL)

(21) Appl. No.: 12/525,835

(22) PCT Filed: Jan. 31, 2008
(86) PCT No.: PCT/IB08/50355

§ 371 Date: Aug. 5, 2009

(30) Foreign Application Priority Data
Feb. 8, 2007 (EP) 07101945.9

(51) Int. Cl. C12Q 1/00; G01N 21/64; G01N 33/24; G01N 33/18

(52) U.S. Cl. 435/4; 422/82.08; 436/25; 436/39; 422/82.11; 385/12; 250/227.11

(57) ABSTRACT

The present invention is directed to a waveguide sensor as well as to an evanescent field induced evanescent field induced sensor system for use in diagnostic housing and an integrated waveguide sensor comprising: a waveguide layer,—capture compounds applied on the upper surface of said waveguide layer for 5 specific bonding to target substances,—a cladding layer surface of said waveguide layer,—a filter which is transmitting for luminescent radiation while absorbs and/or reflects radiation of excitation radiation, wherein the filter is arranged below the lower 10 surface of said cladding layer, at least one detector for sensing luminescent radiation, wherein the detector is arranged below the lower surface of said filter, a substrate that is connected with the detector and comprises the electrical interface of said detector; wherein 15 between the upper surface of the waveguide layer and along at least a lower surface section of the housing a channel is formed for receiving a fluidic probe; and the luminescent radiation is generated by luminescent tag bound to target substances as a result of their excitation by the evanescent field. This provides an improved sensitivity of the evanescent field induced sensor 20 system.

### FIELD OF THE INVENTION

This invention relates to an evanescent field based sensor system. More specifically, the invention relates to a sensor system with evanescent waveguide and integrated detector for use as a chemical or biosensor of detecting biological molecules.

### BACKGROUND OF THE INVENTION

Evanescent luminescence excitation is of great interest in the analytical field, as the excitation is limited to the direct environment of the wave guiding layer. In particular, evanescent field excited fluorescence is a very important technology for biosensors because of its unique sensitivity.

A significant advantage of detecting bound fluorescent molecules that are excited using an evanescent wave is that the emitted fluorescent light can be detected outside the aqueous reaction solution. Most complex biological solutions contain molecules that interfere with fluorescent emission. This means that when a fluorescent molecule is excited it emits a photon and rather than being detected as a signal of a specific binding reaction, the photon is often captured or absorbed by surrounding biological debris or material in the aqueous suspension which is located between the point of emission and the detector. Likewise light can also excite molecules in the aqueous suspension and emit radiation which is not related to the specific binding at the surface. This contribution is often referred to as background radiation or noise radiation and has been associated with conventional assay configurations that emit photons to go through a complex biological matrix prior to detection.

A waveguide biosensor consists generally in the simplest case of a 3-layer system, a first solid substrate, an inorganic wave-conducting layer, and a second solid substrate comprising the sample for assaying, wherein the inorganic wave-conducting layer is sandwiched between the first and second solid substrate. An example of a waveguide biosensor is disclosed in US 2002/0110839 A1 and is incorporated by reference.

The evanescent field waveguide biosensor as disclosed in US 2002/0110839 A1 uses a system of a reaction matrix comprising a waveguide capable of guiding and channelling light and having on the surface of said waveguide a cladding layer having at least one area of depletion in form of nanowells wherein a substance placed within said depletion area can be illuminated by the evanescent wave of light channelled in said waveguide. The manufacture of such a nanowells micro array-cladding layer requires a more complex and more accurate process. Further, the quite small nanowells requires a more accurate and still more complex positioning of the bound molecules in all the wells, which leads to a further drawback. Further, said prior art evanescent field waveguide biosensor has no integrated filter sandwiched between the waveguide layer and the sensor.

Therefore, it is one object of the present invention to provide an evanescent field based optical waveguide sensor suitable for use as a chemical or biosensor of detecting biological molecules, having an improved detection precision of luminescent radiation generated by target substances, can be easily produced and has a low vertical range of manufacture.

### SUMMARY OF THE INVENTION

This object is achieved in providing an evanescent field based waveguide sensor comprising:
a waveguide layer,
capture compounds applied on the upper surface of said waveguide layer for specific bonding to target substances,
a cladding layer contacting arranged on the lower surface of said waveguide layer,
a filter which is transmitting for luminescent radiation while absorbs and/or reflects radiation of excitation radiation, wherein the filter is arranged below the lower surface of said cladding layer,
at least one detector for sensing luminescent radiation, wherein the detector is arranged below the lower surface of said filter, and
a substrate that is connected with the detector and comprises the electrical interface of said detector.

It can be preferred, that the waveguide sensor comprises integrated therein an excitation light source, such as a laser, LED, OLED and/or PLED. Further, the waveguide sensor may comprises integrated therein optical lenses, such as a beam shaper lens and/or a prism.

Most preferred according to the present invention is a an evanescent field based waveguide sensor, wherein at least one filter and at least one detector are integrated in the evanescent field induced sensor/sensor system, so that there is no air space in-between the optical contact of said cladding layer, filter/s and detector/s. This provides an improved sensitivity of the evanescent field induced waveguide sensor and waveguide sensor system because it avoids reflection of the emitted radiation at the air interfaces between the waveguide, cladding layer and detector, which has a negative effect to the sensitivity of the detection of luminescence radiation.

A further object of the present invention is directed to an evanescent field induced sensor system that comprises a housing and an integrated waveguide sensor comprising:
a waveguide layer,
capture compounds applied on the upper surface of said waveguide layer for specific bonding to target substances,
a cladding layer contacting arranged on the lower surface of said waveguide layer,
a filter which is transmitting for luminescent radiation while absorbs and/or reflects radiation of excitation radiation, wherein the filter is arranged below the lower surface of said cladding layer,
at least one detector for sensing luminescent radiation, wherein the detector is arranged below the lower surface of said filter,
a substrate that is connected with the detector and comprises the electrical interface of said detector; wherein
between the upper surface of the waveguide layer and along at least a lower surface section of the housing a channel is formed for receiving a fluidic probe; and the luminescent radiation is generated by luminescent of target substances as a result of their excitation by the evanescent field.

The luminescent radiation is preferably fluorescent light.

A superstrate surrounds and/or contacts at least the upper surface of the waveguide layer. The superstrate is generally water having a refractive index of n 1.33.

It may be preferred, that said waveguide sensor and/or said evanescent field induced sensor system comprises a waveguide layer. Said waveguide layer can be preferably a transparent polymeric waveguide layer. More preferred, said waveguide layer may have a thickness of ≧0.10 μm and ≦0.50 μm and a refractive index n of 1.39 to 1.79.

The refractive index is measured if no other way stated at a temperature of 23° C. and at a wavelength of 632.8 nm.

It may be further preferred, that the lower surface of said waveguide layer is in optical contact or contacts a cladding layer having a refractive index n of 1.29 to 1.69, wherein the material of said waveguide layer and said cladding layer is selected such that the difference of the refractive index Δn of said waveguide layer and said cladding layer is at least Δn 0.1. The cladding layer can be preferably of a transparent polymer material.

A preferred embodiment of the present invention is a waveguide sensor and/or an evanescent field induced sensor system comprising a cladding layer with a low refractive index and a thin layer of a high refractive index waveguide layer, for example a transparent polymeric waveguide layer, spin-coated on top of the upper surface of said cladding layer, wherein the outer upper surface of said waveguide layer possess specific binding compounds to detect at least one specific chemical and/or biochemical substance. Further, sandwiched between the cladding layer and a substrate at least one filter and at least one detector are arranged, wherein the filter is arranged in optical contact above the detector, in order to block excitation radiation and to transmit luminescent radiation, which improves the detection precision of the detector/s.

According to a preferred embodiment of the present invention, the sensor can be of an organic material (OLED/PLED type).

The detector can be mounted on the substrate by means of a suitable bonding material. For example, the detector can be embedded by said bonding material. However, the upper outer surface of the detector is preferably free of said bonding material. It is preferred that the upper outer surface of the detector contacts the lower surface of the filter. Suitable bonding materials are materials having no or practical no auto fluorescence. Such materials are generally known to the expert.

It may be preferred that the detector has a large surface. A large surface of the detector increases the total collection of luminescence emission, while in contrast to that generally an increased surface area contributes to the noise of the sensor. The surface of the detector can be in the range of 0.001 to 1000 mm², preferably 0.01 to 100 mm² and more preferred 0.1 to 10 mm². It is also preferred to have an array of separate detectors on the same substrate. Such an array can be aligned in such a way that every detector collects the radiation coming from a particular biological spot or set of spots for multiplexed detection of targets in a substance.

In order to further improve the detection precision of the detector/s it can be preferred, that the arrangement of the waveguide layer, cladding layer, filter, detector and substrate is in the form of an integrated waveguide sensor having no air space in between. This provides an improved sensitivity and precision of the evanescent field induced sensor system, because it avoids any internal reflection of light and interference effects.

The emission radiation caused by fluorescence molecules is generally not homogeneous in all directions. Further, the main part of the emission radiation light enters the waveguide and cladding layer substrate by a large angle and out coming light of prior art evanescent field based waveguide sensor is diffracted side wards due to total internal reflection. Thus, only a reduced amount of the emission light can reach the detector.

The arrangement of the evanescent field based waveguide sensor according to the present invention, wherein no air space is in-between the waveguide layer, cladding layer, filter and detector to avoid or minimize the internal reflections, so that the detector can receive an increased amount or at least most of the luminescent emission.

Evanescent excitation of luminescence with detection of luminescence in the orthogonal direction to the excitation beam direction avoids the incidence of excitation radiation on the detector in the ideal case. However, due to scattering of the excitation radiation by particles in the excited volume in the sample and/or the waveguide and cladding layer excitation radiation can still hit the detector and create background signal which reduces the sensitivity of detection of targets. By integrating the detector with the waveguide according to the preferred embodiment the contribution of other external radiation is minimized. Therefore it is subject of the present invention to include a filter in the light path between the waveguide layer and the detector, preferably between the cladding layer and the detector surface. Filters can be used based on different physical operating principles, like absorption, reflection and interference. In the preferred arrangement the filter selectivity can be adjusted to the requirements of the application. In certain cases a lower selectivity can be tolerated which allows the use of absorption filters, based on solid solutions of dyes in a transparent matrix have a low vertical range of manufacture but relatively inferior filter characteristic compared to dichroic interference filters. Optical polymer filters useful according to the present invention are for instance dye doped polymer layers, like polydimethylsiloxane (PDMS) layer/s, doped with Sudan dyes.

However, it is preferred that the filter is highly translucent for the emission radiation of fluorophores and is not translucent or poor translucent for the excitation radiation. The selectivity in the transmission of the emission radiation over the excitation radiation should be at least a factor 10. However, a factor for the selectivity in the transmission of the emission radiation over the excitation radiation between 100 and 1000000 is preferred. Thus, the ratio of the transmission of the emission radiation over the excitation radiation can be in the range of ≧10:1 to 1.000.000, preferably ≧100:1, further preferred ≧1000:1, more preferred ≧10000:1 and most preferred ≧100000 to 1 or ≧1000000:1.

In general, the difference of the maxima of the excitation radiation and the maxima of the luminescent radiation is about 20 nm to 30 nm, where the emission wavelength is shifted to the red with respect to the excitation. Therefore, it is preferred, that the filter is selected such that edge of the transmission spectrum shows a sharp transition from absorbing to transmissive in the wavelength region between the excitation and emission radiation. A suitable filter can show a high absorbance up to 670 nm and a high transmission from 690 nm and higher for a dye which is excited with radiation of 660 nm and has a maximum emission at 700 nm.

The components of the evanescent field induced sensor system comprising the waveguide layer, the cladding layer and the substrate can be all of polymer material and preferably are all of transparent organic polymer/s. Further, the detector and/or the filter can be of an organic material, preferably an organic polymer. Furthermore, the waveguide sensor and/or the evanescent field induced sensor system can be of an organic material, preferably an organic polymer. This makes the waveguide sensor and/or the evanescent field induced sensor system more stable, due to the better matching of the thermo-mechanical properties. Further, an all organic polymer waveguide sensor and/or evanescent field induced sensor system has an increased flexibility compared to an waveguide sensor and evanescent field induced sensor system with inorganic layer structure.

In the sense of the present invention the term “polymer” includes thermoplastic, thermosetting and/or structurally cross-linked plastic.

In order to achieve a sufficient intensive evanescent field the adjustment of the waveguide layer thickness and the difference of the refractive index Δn of said waveguide layer and said cladding layer is important.

The thickness of the waveguide layer may be selected so that X is in the range of 1 to 9, preferably in the range of 1.2 to 6, more preferably 1.5 to 4.5 and most preferably 2 to 3.5, whereby d is calculated based on the equation:

$$X = \frac{d {n}_{2} 2 π }{λ}$$

in which d is the thickness of the waveguide layer in nm, n₂ is the refractive index of the waveguide layer and λ is the wavelength in nm, wherein the wavelength is in the range of 360 nm to 1000 nm, preferably 400 nm to 800 nm and more preferably 600 nm to 750 nm.

The thickness of the thin waveguide layer can be of ≧0.12 μm and ≦0.40 μm, preferably ≧0.14 μm and ≦0.30 μm, more preferably ≧0.16 μm and ≦0.28 μm and most preferably ≧0.18 μm and ≦0.24 μm for a wavelength of 633 nm.

However, it can be beneficial that the smaller the difference of the refractive index Δn of said waveguide layer and said cladding layer the larger can be the thickness of the waveguide.

It may be preferred that the thickness of the waveguide layer can be of ≧0.13 μm and ≦0.29 μm. An increased intensive evanescent field of the evanescent field induced sensor system can be obtained for the waveguide layer with a thickness of ≧0.17 μm and ≦0.22 μm.

The thickness of the cladding layer can be of ≧2 μm and ≦5 mm. However, it can be preferred that the thickness of the cladding layer is of ≧20 μm and ≦3 mm and more preferred of ≧50 μm and ≦1.5 mm. The thickness of the cladding layer can also be of ≧100 μm and ≦500 μm.

It can be preferred that he lower surface of said waveguide layer completely contacts the upper surface of the cladding layer.

The other critical feature is the refractive index difference of Δn 0.1 to Δn 0.5, preferably of Δn 0.2 to Δn 0.4 and more preferably of Δn 0.25 to Δn 0.35 with respect to said waveguide layer and the cladding layer in order to obtain a sufficient high evanescent intensity for qualitative and/or quantitative determination of chemical or biochemical compounds with the evanescent field induced sensor system according to the present invention.

Further, it is important that the refractive index n of the waveguide layer is higher than the refractive index n of the cladding layer, which contacts the lower surface of said waveguide.

It may be preferred, that said waveguide layer has a coupling grating recess structure for enhancing the coupling of the light wave into said waveguide layer. The waveguide layer can have at least one recess and/or at least one heightening on the outer upper surface of said waveguide. Further the waveguide layer can have at least one recess and/or at least one heightening on the lower upper surface of said waveguide contacting the cladding layer, wherein the heightening/s engage with a positive fit into the cladding layer contacting said polymer waveguide and the cladding layer engage with a positive fit into the lower upper surface of said waveguide. The recess can be important for enhancing the coupling of the light wave into said waveguide layer, wherein the depth of said recess is less than the thickness of the said waveguide layer. It is preferred that a grating structure of a plurality of recesses for enhancing the coupling of the light wave into said waveguide layer is formed on the upper and/or lower surface of the waveguide layer.

According to a preferred embodiment of the present invention the top surface of the heightening/s does not exceed the upper outer surface of the waveguide layer.

It can be preferred that a surface area of said waveguide layer of at least 5% to 95%, preferably 5% to 25%, more preferably 5% to 15% of the upper outer surface and/or lower inner surface of said waveguide layer comprises a grating structure of a plurality of recesses for enhancing the coupling of the light wave into said waveguide layer.

The grating period can be from 250 nm to 950 nm, more preferred from 300 nm to 750 nm and most preferred from 350 nm to 450 nm. The grating exhibits one periodicity only, i.e. is mono diffractive. However it can be preferred that the grating exhibits more than one periodicity, such as two or three periodicities and/or a gradual variation of the periodicity.

According to the present invention it can be preferred, that the upper surface of the waveguide layer is neither contacted by a cladding layer or substrate. Thus, it is preferred that no nanowells micro arrays are arranged on the upper surface of the waveguide layer and/or in optical contact with the waveguide layer.

It can be an advantage of the evanescent field induced sensor system according to the present invention that the above mentioned selection of defined material characteristics provide an optical biosensor which has a high performance for detecting specific chemical and/or biochemical substances with high qualitative and/or quantitative precision.

A further advantage is, that the evanescent field induced sensor system according to the present invention has a low vertical range of manufacture, since the waveguide can be applied by spin coating or printing to the moulded or cast polymeric substrate. However, it is also possible to cast or mould the polymeric substrate on the spin coated or printed polymeric waveguide.

The evanescent field induced sensor system according to the present invention can be used also for the surface Plasmon resonance based detection with respect to the presence of chemical- and/or bio-molecules on the surface of the waveguide layer. In this case the upper outer surface of the wave guide layer is covered with a thin metal layer, preferably Au.

A benefit of the evanescent field induced sensor system according to the present invention comprising polymer materials is that for example that polymer materials match the chemical and thermo-mechanical properties. Thus, failure during the various treatments which are required for the application of bio-sensing on top of the waveguide is significantly reduced compared to evanescent field induced sensor systems having an inorganic waveguide layer. Problems may arise of inorganic waveguide layers are due to an inherent difference in thermal expansion of the inorganic waveguide layer and the contacting substrate layer, for example a cladding layer, which leads to stresses in said layer and a high mechanical stress in the interface, which leads to cracking and delaminating.

The waveguide layer, the cladding layer and the substrate according to the present invention can preferably have a planar form.

Within the scope of this invention, the evanescent field induced sensor system can be in form of a strip, a plate, a round disc or of any other geometrical form. The chosen geometrical form is not crucial and can be governed by the intended sensor use. It may, however, also be used as an independent element, spatially separated from the source of excitation light and from the optoelectronic detection system.

The specific binding compounds to detect at least one specific chemical and/or biochemical substance can be bonded direct to the outer upper surface of the waveguide layer or contacted to the outer upper surface of the waveguide layer, for example by means of adsorption, and/or can be stuck to the outer upper surface of the waveguide layer, for example by direct chemical reaction or via a chemical linker molecules. This can be done in a patterned fashion and for a multitude of different kinds of specific probes by printing techniques.

In order to have a minimum vertical range of manufacture it is most preferred that the evanescent field induced sensor system according to the present invention does not comprise a waveguide layer with a cladding layer on top having at least one area of depletion in form of nanowells, wherein bound molecules are placed within said depletion area as detecting material. Thus, the evanescent field induced sensor system according to the present invention may exclude a waveguide layer capable of guiding and channeling light and having on the top surface of said waveguide layer a cladding layer having at least one area of depletion wherein a substance placed within said depletion area can be illuminated by the evanescent wave of light channeled in said waveguide layer.

Further, it may be preferred that the grating or recess is free of detecting material. However, the detecting material may be applied on the grating structure.

Typically, the attenuation of the guided light wave in the waveguide layer is less than 0.5 dB/cm, preferably less than 0.01 dB/cm measured with a light source emitting at 633 nm, thereby resulting in a long distance of the guided beam and a low scattering of the guided wave into the media surrounding it. In particular, it is preferred under these conditions to guide the TE and/or TM mode.

The thickness of the waveguide layer according to the present invention is small enough so that only one TM mode and/or one TE mode can propagate in the waveguide layer.

The polymer material for the cladding layer is preferably selected from the group comprising transparent polymers such as olefines, cyclic olefines, acrylates, methacrylates, ethers, esters, urethanes, ether-esters, ether-urethanes, urethane acrylates, enols, etc. and partially or perfluorinated analogons of these materials, silicones, silicone-acrylates and -methacrylates.

More preferred as cladding layer transparent polymer material/s are halogenated polymers, in particular fluorinated or perfluorinated polymers. Thus, most preferred are halogenated acrylates, halogenated methacrylates, acrylates with perfluorinated side chains and/or methacrylates with perfluorinated side chains as well as copolymers thereof, for example with low refractive index n_{D}=1.37-1.41.

The transparent material of the cladding layer has a lower refractive index than the waveguide layer, i.e. a refractive index n_{D} of at most 1.69.

Most preferred, the cladding layer material of the substrate is cross-linked.

A suitable waveguide layer material is typically any kind oftransparent polymeric material with a higher refractive index than the cladding layer. It is preferred to use a transparent polymer that has an optical refractive index as high as possible.

Also, it may be preferred that the wave guide layer material can be optically processed in as simple manner as possible, for example spin coating on top of the upper outer surface of the cladding layer.

Most preferably, the cladding layer material should be highly transparent at least at the fluorescence emission wavelength and shows preferably no auto-fluorescence.

In the sense of the present invention the term “transparent waveguide layer material” or “wave guide layer material” includes thermoplastic, thermosetting and/or structurally cross-linked plastic/s having all a higher refractive index than the cladding layer, i.e. a refractive index n_{D} of at least 1.39.

The material for the waveguide layer is preferably selected from the group comprising homocyclic and/or heterocyclic aromatics, halogenated and/or sulphur containing polymers. Bromium and/or sulphur containing polymers, in particular bromium and/or sulfur containing polymers with limited delocalised pi-system are preferred.

More preferred materials for the waveguide layer are Poly(penta-bromophenyl methacrylate (n_{D}=1.71), Poly(vinylphenylsulfide) (n_{D}=1.657), Bisphenol-S based epoxides and/or -acrylates or such like.

The outer upper surface of said waveguide layer possesses specific binding compounds to detect at least one specific chemical and/or biochemical substance.

The surface of said waveguide layer can be treated and covered with specific, for example adhesion, layers to bind for instance biomolecules, like antibodies or cDNA strands for selective binding or hybridization of biological targets in sample liquids which are directed over the treated surface of the device for the analysis of the liquid of interest. The presence of bound biomolecules is detected for instance by the fluorescence excited by the evanescent field of the waveguide of the sensor system according to the present invention.

Within the scope of this invention the term “sample” or “probe” or “fluidic sample” or “fluidic probe” or “superstrate” shall be taken to mean the entire solution to be assayed, which may contain a substance to detect an analyte. The detection can be made in a single-step or multistep assay in the course of which the surface of the waveguide layer of the evanescent field induced sensor system according to the present invention is contacted with one or more solutions. At least one of the solutions employed can contain a substance having luminescence properties, which can be detected in the practice of this invention.

If a substance having luminescence properties is already adsorbed on the upper waveguide surface, then the sample may also be free from luminescent components.

The sample can contain further constituents, typically pH buffers, salts, acids, bases, surface-active substances, viscosity-influencing modifiers or dyes. In particular, a physiological saline solution can be used as solvent. If the luminescent constituent itself is liquid, then the addition of a solvent can be dispensed with.

The sample may further contain a biological medium, for example egg yolk, a body fluid or constituents thereof, in particular blood, serum, plasma or urine. Furthermore, the sample may consist of surface water, solutions of extracts of natural or synthetic media such as soil or parts of plants, bioprocess broths or synthesis broths.

The sample can either be undiluted or used additionally with a solvent. Suitable fluids are solvents such as water, aqueous buffer and protein solutions and organic solvents.

Suitable organic solvents are alcohols, ketones, esters, and aliphatic hydrocarbons. It is preferred to use water, aqueous buffers or a mixture of water and a water-miscible organic solvent. The sample can, however, also contain constituents that are insoluble in the solvent, for example pigment particles, dispersants, natural and synthetic oligomers or polymers. In this case the sample is in the form of an optically turbid dispersion or emulsion.

Suitable luminescent compounds are luminescent dyes having a luminescence in the wavelength range from 360 nm to 1000 nm, typically including rhodamines, fluorescein derivatives, coumarin derivatives, distyryl biphenyls, stilbene derivatives, phthalocyanines, naphthalocyanines, polypyridyl-ruthenium complexes such as tris(2,2′-bipyridyl)ruthenium chloride, tris(1,10-phenanthroline) ruthenium chloride, tris(4,7-diphenyl-1,10-phenanthroline) ruthenium chloride and polypyridyl-phenazine-ruthenium complexes, platinum-porphyrin complexes such as octaethyl-platinum-porphyrin, long-life europium and/or terbium complexes or cyanine dyes, so-called quantum-dots, such as GaN, or InP or other. Suitable for analyses in blood or serum are dyes having absorption and emission wavelengths in the range from 360 nm to 1500 nm.

Particularly suitable luminescent compounds are dyes such as fluorescein derivatives, which contain functional groups with which they can be covalently bonded, for example fluorescein isothiocyanate.

The preferred luminescence is fluorescence.

The luminescent dyes eligible for use may also be chemically bonded to polymers or to one of the binding partners in biochemical affinity systems, e.g. antibodies or antibody fragments, antigens, proteins, peptides, receptors or their ligands, hormones or hormone receptors, oligonucleotides, DNA strands and RNA strands, DNA or RNA analogs, binding proteins such as protein A and G, avidin or biotin, enzymes, enzyme cofactors or 0 inhibitors, lectins or carbohydrates. The covalent luminescent labelling last mentioned is the preferred utility for reversible or irreversible (bio)chemical affinity assays. It is further possible to use luminescent-labelled steroids, lipids and chelators. Intercalating luminescent dyes are also of particular interest for hybridisation assays with DNA strands or oligonucleotides, especially if—like different ruthenium complexes—they exhibit enhanced luminescence in the intercalation. If these luminescent-labelled compounds are brought into contact with their affinity partners immobilised on the surface of the evanescent field induced sensor system according to the present invention, then the binding can be determined quantitatively from the measured intensity of luminescence. A quantitative determination of the analyte is also possible by measuring the change in luminescence when the sample interacts with the luminophores, for example in the form of luminescence quenching with oxygen or of luminescence enhancement by conformation modifications of proteins.

It is preferred to use coherent light for the luminescence excitation, more particularly laser light of wavelength 300 to 1100 nm, more particularly still of 400 to 850 nm and, most preferably, of 540 to 700 nm.

Lasers, which may suitably be used, are dye lasers, gas lasers, solid lasers and semiconductor lasers. Where necessary, the emission wavelength can also be doubled by nonlinear crystal optics. The beam can also be still further focused by optical elements, polarised, or attenuated by grey filters. Particularly suitable lasers are argon-ion lasers and helium-neon lasers which emit at wavelengths between 457 nm and 514 nm and, respectively, between 543 nm and 633 nm. Very particularly suitable lasers are diode lasers or frequency-doubled diode lasers of semiconductor material that emit at a fundamental wavelength between 630 nm and 1100 nm, as they permit a substantial miniaturisation of the entire sensor system on account of their small dimensions and low power consumption. However diode lasers with about 405 nm and sufficient power can be used also.

In the process of this invention the sample can be brought into contact with the evanescent field induced sensor system in the immobile state as well as guided continuously over it, and the cycle can be open or closed.

A specific embodiment of the process consists in immobilising the substances having luminescent properties used for detecting the analyte direct at the surface of the waveguide layer. The substance having luminescent properties can be, for example, a luminophore, which is bound to a protein and which can thereby be excited to luminescence in this manner at the surface of the waveguide layer. If a partner having affinity for the protein is guided over this immobilised layer, then the luminescence can be modified and the amount of said partner could be determined in this manner. In particular, both partners of an affinity complex can also be labelled with luminophores so as to be able to effect the determination of concentrations from the energy transfer between the two, e.g. in the form of luminescence quenching.

Another preferred embodiment of the process for carrying out chemical or biochemical affinity assays consists in immobilising on the surface of the evanescent field induced sensor system, i.e. upper outer surface of the waveguide, a specific binding partner as chemical or biochemical detector substance for the analyte itself or for one of the binding partners. The assay can be a single-step or multistep assay in the course of which, in successive steps, one or more than one solution containing binding partners for the detector substances immobilised on the surface of the evanescent field induced sensor system according to the present invention is guided, the analyte becoming bound in one of the partial steps. Binding luminescent-labelled participants in the affinity assay effects the detection of the analyte. The luminescent-labelled substances used may consist of one or more than one binding partner of the affinity assay, or also of an analogue of the analyte provided with a luminophore. The sole criterion is that the presence of the analyte leads selectively to a luminescence signal or selectively to a change in the luminescence signal.

The immobilisation of the detector substances may typically be carried out by hydrophobic absorption or covalent bonding direct on the upper outer waveguide surface or after chemical modification of the surface, for example by silanisation or applying a polymer layer. In addition, a thin interlayer consisting e.g. of SiO₂ can be applied as adhesion-promoting layer direct to the upper outer waveguide surface to facilitate the immobilisation of the detector substances direct on the waveguide.

Suitable detector substances are typically antibodies for antigens, binding proteins such as protein A and G for immunoglobulins, receptors for ligands, oligonucleotides and single strands of RNA and DNA for their complementary strands, avidin for biotin, enzymes for enzyme substrates, enzyme cofactors or inhibitors, lectins for carbohydrates. Which of the respective affinity partners is immobilised on the surface of the evanescent field induced sensor system according to the present invention will depend on the architecture of the assay.

The assay itself can be a single-step complexing process, for example a competitive assay, or also a multistep process, for example a sandwich assay.

In the simplest case of the competitive assay, the sample which contains the analyte in unknown concentration as well as a known amount of a compound that is similar except for luminescent labelling is brought in to contact with the surface of the evanescent field induced sensor system according to the present invention, where the luminescent labelled and unlabelled molecules compete for the binding sites at their immobilised detector substances. A maximum luminescence signal is achieved in this assay configuration when the sample contains no analyte. With increasing concentration of the substance to be detected, the luminescence signals under observation become lower.

In a competitive immunoassay it does not necessarily have to be the antibody, which is immobilised: the antigen too can be immobilised on the surface of the evanescent field induced sensor system according to the present invention as detector substance. Usually it is immaterial which of the partners is immobilised in chemical or biochemical affinity assays. This is a basic advantage of luminescence-based assays over methods such as surface Plasmon resonance or interferometry, which are based on the change in adsorbed mass in the evanescent field of the waveguide layer.

Further, in the case of competitive assays the competition does not need to be limited to binding sites at the surface of the evanescent field induced sensor system according to the present invention. For example, a known amount of an antigen can also be immobilised on the surface of said sensor and then brought into contact with the sample which contains an unknown amount to be detected of the same antigen as analyte as well as luminescent-labelled antibodies. In this case the competition between antigens that are immobilised on the surface and present in solution takes place for binding of the antibodies.

The simplest case of a multistep assay is a sandwich immunoassay in which a primary antibody is immobilised on the surface of the evanescent field induced sensor system according to the present invention. The binding of the antigen to be detected and of the luminescent-labelled secondary antibody used for carrying out the detection to a second epitope of the antigen can be effected either by successive contacting with the solution containing the antigen and a second solution containing the luminescent-labelled antibody, or by combining these two solutions beforehand, so that, finally, the partial complex consisting of antigen and luminescent-labelled antibody is bound.

Affinity assays may also comprise further additional binding steps. For example, in the case of sandwich immunoassays protein A, which specifically binds immunoglobulins which then act as primary antibodies in a subsequent sandwich assay, which is carried out as described above, at their so called F_{c} part, can be immobilised on the surface of the evanescent field induced sensor system according to the present invention in a first step.

There is a whole host of further types of affinity assays, typically using the known avidin-biotin affinity system.

It is furthermore possible to use the surface of the evanescent field induced sensor system according to the present invention not only for single use but also to regenerate it. Under suitable conditions, for example low pH, elevated temperature, using organic solvents or so-called chaotropic reagents (salts), it is possible to dissociate the affinity complexes selectively without substantial impairment of the binding capacity of the immobilised detector substances. The exact conditions are strongly dependent on the particular affinity system.

Another essential embodiment of the process consists on the one hand in limiting the production of the signal—in the case of back-coupling this also applies to signal detection—to the evanescent field of the waveguide and, on the other, in the reversibility of the affinity complex formation as equilibrium process. Using suitable rates of flow in a continuous flow system it is possible to monitor in real time the binding or desorption or dissociation of bound luminescent-labelled affinity partners in the evanescent field. The process is therefore suitable for kinetic studies for determining different association or dissociation constants or also for displacement assays.

The most important design criteria are the intensity of the evanescent field at the surface of the waveguide. This intensity is determined by the refractive indices of the waveguide layer (n2), the substrate (n1) and the superstrate (n3), the thickness of the waveguide layer (6).

This intensity decays exponentially with increasing distance from the wave guide surface. For optimize the embodiment one can take the average intensity of the evanescent field in a range of the expected thickness of adsorbed biomolecules in which the dye molecules are to be excited by the same field.

Preferred embodiments of an integrated waveguide sensor of the evanescent field induced sensor system according to the present invention are outlined below: waveguide layer:
poly-pentabromophenylacrylate (Aldrich);
Irgacure 184 (Ciba) was added to the monomer to enable photo polymerization;
   cladding layer:
low index substrate: 2,2,3,3,4,4,5,5-octafluoro-1,6-hexanedioldimethacrylate (ABCR); filter:
solid solution of dye molecules in polymer matrix (e.g. Sudan II in PDMS) sensor:
optical sensor amorphous silicon, or (low-temperature) polysilicon ((LTPS) thin film sensor
   substrate:
polymer or glass.

The refractive indices of the polymeric cladding layer and the polymeric waveguide layer are about 1.44 and 1.70 respectively. The final thickness of the polymeric cladding layer is about 2 μm and that of the polymeric waveguide layer is about 210 nm.

The evanescent field induced sensor system according to the present invention comprises a housing. The housing receives the integrated waveguide sensor.

It may be preferred that the housing is removable connected with the integrated waveguide sensor. This provides an evanescent field induced sensor system at which the housing can be reused where else the integrated waveguide sensor can be disposed.

Thus, it may be preferred to integrate in the housing the excitation source, for example a laser, a beam shaper and/or a prism.

The housing can be an integral part of a medical apparatus, a diagnostic apparatus, read out device, or surgical instrument, for example an endoscope and the like.

### BRIEF DESCRIPTION OF THE DRAWINGS

FIG. 1 shows a second evanescent field induced sensor system according to the present invention;

FIG. 2 shows a third evanescent field induced sensor system according to the present invention;

FIG. 3 shows a fourth evanescent field induced sensor system according to the present invention;

### DETAILED DESCRIPTION OF THE EMBODIMENTS

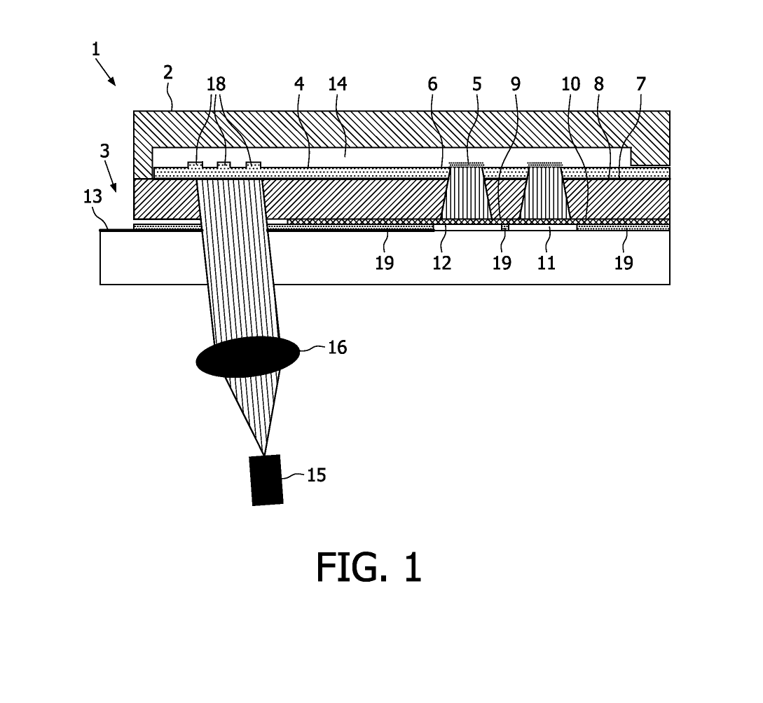

FIG. 1 shows an evanescent field induced sensor system 1 useful in diagnostic applications with an housing 2. The housing 2 is arranged on top of an integrated waveguide sensor 3. The integrated waveguide sensor 3 comprises a polymer waveguide layer 4 of polyethersulfone (PES) with a thickness of 250 nm and a refractive index of n2=1.65 on a cladding layer 7 of polymethylmethacrylate (PMMA) with a thickness of 1 mm and a refractive index of n3=1.49. On top of the upper outer surface 6 of the polymer waveguide layer 4 capture compounds 5 are arranged to detect a specific chemical substance. On the upper outer surface of the polymer waveguide layer 4 a grating structure of a plurality of recesses 18 for enhancing the coupling of the light wave into said transparent polymer waveguide are arranged. Alternatively, the grating structure can also be present at the interface between the cladding layer and the wave guiding layer. Between the upper outer surface of the polymer waveguide layer 4 and the housing 2 a channel 14 is formed for receiving an aqueous probe (n1≈1.33). The upper outer surface of the cladding layer 7 contacts the lower surface 8 of said waveguide layer 4. Further, a polymer filter 9 or as an alternative a dichroic filter which is transmitting for fluorescent radiation above 680 nm is arranged below and contacts the lower surface 10 of said cladding layer 7. Two detectors 11 for sensing the fluorescent radiation generated by fluorescent tag bound to target substances as a result of their excitation by the evanescent field are arranged on the upper surface of a substrate 13 and below the lower surface 12 of said filter 9. The detectors 11 are mounted by means of a (planarizing) bonding material 19 to surface 12. The arrangement of the waveguide layer 4, cladding layer 7, filter 9, detector 11 and substrate 13 is in the form of an integrated waveguide sensor 3 having no air space in between. This provides an improved sensitivity of the evanescent field induced sensor system 1, because it avoids any air interference between the waveguide 4, cladding layer 7 and detector 11, which may have a negative effect to the luminescence radiation. Further, the filter 9 improves the luminescence collection efficiency of the detector 11. The excitation radiation is generated by a solid state laser source 15 with a wave length of 660 nm. The excitation radiation is projected on the grating area of the waveguide by a set of lenses 16 which can include a beam shaper, diaphragm and collimating lens.

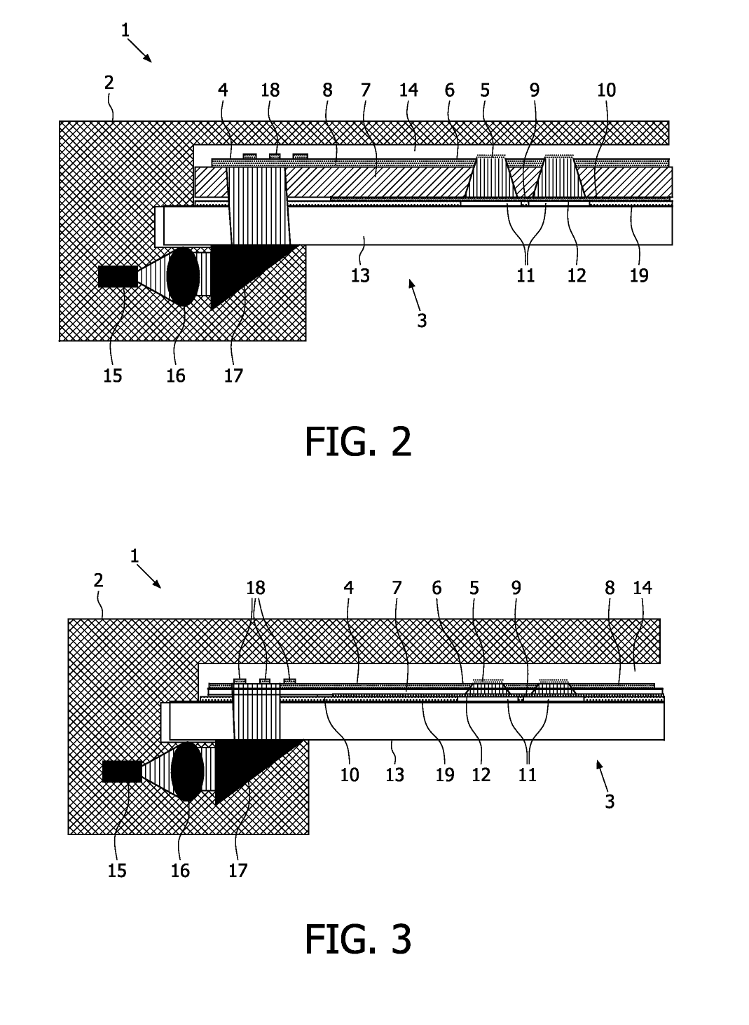

FIG. 2 shows an evanescent field induced sensor system 1 useful in diagnostic applications with an housing 2, wherein the housing 2 is removable connected with the integrated waveguide sensor 3. This allows a reuse of the housing 2 and the integrated sensor 3 can be toss away after use. Further, the laser source 15, the beam shaper lens 16 and the prism 17 are arranged in the housing 2, which facilitates and speeds up the use of the evanescent field induced sensor system 1. The integrated waveguide sensor 3 comprises an inorganic waveguide layer 4 of Ta₂O₅ with a thickness of 130 nm and a refractive index of n2=2.15 on a cladding layer 7 of cycloolefin(co)polymer (COP) with a thickness of 0.6 mm and a refractive index of n3=1.53. On top of the upper outer surface 6 of the polymer waveguide layer 4 capture compounds 5 are arranged to detect a specific chemical substance. Between the upper outer surface of the polymer waveguide layer 4 and the housing 2 a channel 14 is formed for receiving an aqueous probe (n1≈1.33). The upper outer surface of the cladding layer 7 contacts the lower surface 8 of said waveguide layer 4. Further, a filter 9 such as an inorganic multilayer stack on a polymeric substrate layer, which is transmitting for fluorescent radiation above 650 nm, is arranged below and contacts the lower surface 10 of said cladding layer 7. Two detector arrays 11 for sensing the fluorescent radiation generated by fluorescent tag bound to target substances as a result of their excitation by the evanescent field are arranged on the upper surface of a substrate 13 and below the lower surface 12 of said filter 9. The detectors 11 are mounted by means of a bonding material 19. The arrangement of the waveguide layer 4, cladding layer 7, filter 9, detector 11 and substrate 13 is in the form of an integrated waveguide sensor 3 having no air space in between. This provides an improved sensitivity of the evanescent field induced sensor system 1, because it avoids reflection of the emitted radiation at the air interfaces between the waveguide 4, cladding layer 7 and detector 11, which has a negative effect to the sensitivity of the detection of luminescence radiation. Further, the filter 9 improves the luminescence collection efficiency of the detector 11. The excitation radiation is generated in the housing 2 by a laser source 15 with a wave length of 633 nm. The excitation radiation is collimated by a beam shaper lens 16 and turned around 90° by a prism 17, which allows a flat construction of the evanescent field induced sensor system. The housing 2 can be an integral part of an medical apparatus, read out device, diagnostic apparatus or surgical instrument (not shown).

FIG. 3 shows an evanescent field induced sensor system 1 according to FIG. 2 with the exception that the cladding layer 7 has a very thin layer thickness, wherein the cladding layer has a thickness of 0.01 mm to 0.2 mm. This improves further the efficiency of detector capture of fluorescent light generated by the fluorescent tag bound to target substances as a result of their excitation by the evanescent field. More importantly, this allows a different manufacturing and assembling process, based on foil technology and/or spin- and roller coating technology. In this way multiple sensors can be processed on a single substrate (wafer based or reel-to-reel).

The evanescent field induced sensor system 1 with said filter 9 has the advantage compared by the same evanescent field induced sensor system but with the exception that no filter is used, that the detector sensitivity can be improved since noise radiation can be eliminated.

According to the present invention it may be preferred that the evanescent intensity, i.e. TM field fraction in the superstrate water, over a distance of 20 nm perpendicular to the outer upper surface of said evanescent field induced sensor system there from should be adjusted to be in the range of 0.002 to 0.01, preferably in the range of 0.003 to 0.008, more preferred in the range of 0.004 to 0.007.

The TM field can be calculated according to W. Lukosz and K. Tiefenthaler “sensitivity of grating couplers as integrated-optical chemical sensors”, J. Opt. Soc. Am. B6(2) (1989) pp. 209-220 in comparison to the benchmark of the evanescent field induced sensor system (see FIG. 2) consisting of a waveguide of Ta₂O₅ with a refractive index of n_{D} 2.13 and a Zeonex 280 substrate*¹ with a refractive index of n_{D} 1.53, whereby the superstrate is water with a refractive index of n_{D} 1.33. *¹: Zeonex 280 can be obtained from Nippon Zeon Co., LTD.

The fraction of the electric field in the superstrate of the waveguide may be preferably calculated using the equation:

$${I}_{TM}^{sup} = \frac{( {ɛ}_{2} - {n}_{eff}^{2} ) {ɛ}_{2}}{( {ɛ}_{2} - {n}_{1} ) {n}_{eff}^{2}} \frac{1}{{q}_{1} ( {n}_{eff} ) [ d \cdot {k}_{0} + δ {z}_{1} ( {n}_{eff} ) + δ {z}_{3} ( {n}_{eff} ) ]}$$

where δz₁ is the penetration depth in the superstrate and δz₃ is the penetration depth in the substrate, given by:

$$δ {z}_{1} ( {n}_{eff} ) = \frac{1}{{q}_{1} ( {n}_{eff} ) [ {n}_{eff}^{2} ( 1 / {ɛ}_{1} + 1 / {ɛ}_{2} ) - 1 ]}$$

$$δ {z}_{3} ( {n}_{eff} ) = \frac{1}{{q}_{3} ( {n}_{eff} ) [ {n}_{eff}^{2} ( 1 / {ɛ}_{3} + 1 / {ɛ}_{2} ) - 1 ]}$$

In these equation q₁ is the imaginary part of the wave vector in the superstrate and q₃ the imaginary part of the wave vector in the substrate given by:

q₁(n_{eff})=\sqrt{n_{eff}²−∈₁}

q₃(n_{eff})=\sqrt{n_{eff}²−∈₃}

The quantity n_{eff} describes the effective refractive index of the propagating mode. The value for the propagation constant of the TM mode can be found by solving the following equation:

k_{z}(n_{eff})k₀d−φₘ₁(n_{eff})−φₘ₃(n_{eff})−mπ=0

Here, m=0, 1, 2 . . . is the mode number and the phase functions φₘ₁ and φₘ₃ are given by:

$${φ}_{m 1} ( {n}_{eff} ) = {tan}^{- 1} ( \frac{{ɛ}_{2} {q}_{1} ( {n}_{eff} )}{{ɛ}_{1} {k}_{z} ( {n}_{eff} )} )$$

$${φ}_{m 3} ( {n}_{eff} ) = {tan}^{- 1} ( \frac{{ɛ}_{2} {q}_{3} ( {n}_{eff} )}{{ɛ}_{3} {k}_{z} ( {n}_{eff} )} )$$

$$with$$

$${k}_{z} ( {n}_{eff} ) = \sqrt{{ɛ}_{2} - {n}_{eff}^{2}}$$

In the equations the dielectric constants of the superstrate, wave guide layer and substrate are denoted by ∈₁, ∈₂ and ∈₃ respectively, k₀ is the wave vector in vacuum and d is the thickness of the wave guide layer.

In one of its aspects the invention relates to a process for detecting luminescence with an evanescent field induced sensor system according to the present invention by bringing a liquid sample into contact with the upper surface of the waveguide layer or upper surface of the bonding material attached to the upper surface of the waveguide layer, and measuring the luminescence produced by substances having luminescence properties in the sample, or by substances having luminescence properties immobilised on said waveguide, optoelectronically, wherein the excitation light is coupled into the said waveguide and traverses the wave guiding layer, whereby the substances having luminescence properties are excited to luminescence in the evanescent field of the wave guiding layer.

Detectors for the detection of the evanescently excited luminescence are for example photodiodes, photocells, photomultipliers, charge-coupled device (CCD) arrays and detector arrays, for example CCD cameras, may suitably be used. Useful detectors have a photosensitive element that generates a voltage or current when exposed to light.

However, most preferred are silicon-based detectors because of the low manufacturing cost. One example for a preferred silicon-based detector is an α-Si diode sensor.

Also preferred are functional polymer-based detectors because of the even lower manufacturing cost and the processing compatibility with the other layers of the total sensor system.

In another of its aspects, the invention relates to the use of the evanescent field induced sensor system according to the present invention for the quantitative determination of chemical or biochemical compounds such as antibodies or antigens.

Yet another utility of the evanescent field induced sensor system according to the present invention is for the quantitative determination of receptors or ligands, oligonucleotides, strands of DNA or RNA, DNA or RNA analogs, enzymes, enzyme substrates, enzyme cofactors or inhibitors, lectins and carbohydrates.

In a further aspect, the invention relates to the use of the evanescent field induced sensor system according to the present invention for the selective quantitative determination of luminescent constituents in optically turbid fluids.

Optically turbid fluids may typically be biological fluids such as egg yolk, body fluids such as blood, serum or plasma, and also samples emanating from environmental analysis, including surface water, dissolved soil extracts and dissolved plant extracts. Suitable fluids are also the reaction solutions obtained in chemical production, in particular dye solutions or reaction solutions originating from the production of luminescent, such as fluorescent, whitening agents. Also suitable are all types of the dispersions and formulations typically used in the textile industry, provided these contain one or more than one luminescent component.

The evanescent field induced sensor system according to the present invention can also be used for quality safeguarding.

In summary the evanescent field induced sensor system according to the present invention can for example be used for:
chemical or biological analysis, comprising analysis of biological fluids such as egg yolk, blood, serum or plasma;
environmental analysis, comprising analysis of water, dissolved soil extracts and dissolved plant extracts;
reaction solutions, dispersions and/or formulations analysis, comprising analysis in chemical production, in particular dye solutions or reaction solutions; and/or
quality safeguarding analysis.

To provide a comprehensive disclosure without unduly lengthening the specification, the applicant hereby incorporates by reference each of the patents and patent applications referenced above.

The particular combinations of elements and features in the above detailed embodiments are exemplary only; the interchanging and substitution of these teachings with other teachings in this and the patents/applications incorporated by reference are also expressly contemplated. As those skilled in the art will recognize, variations, modifications, and other implementations of what is described herein can occur to those of ordinary skill in the art without departing from the spirit and the scope of the invention as claimed. Accordingly, the foregoing description is by way of example only and is not intended as limiting. The invention's scope is defined in the following claims and the equivalents thereto. Furthermore, reference signs used in the description and claims do not limit the scope of the invention as claimed.

1. An evanescent field based waveguide sensor (3) comprising:
a waveguide layer (4),
capture compounds (5) applied on the upper surface (6) of said waveguide layer (4) for specific bonding to target substances,
a cladding layer (7) contacting arranged on the lower surface (8) of said waveguide layer (4),
a filter (9) which is transmitting for luminescent radiation while absorbs and/or reflects radiation of excitation radiation, wherein the filter (9) is arranged below the lower surface (10) of said cladding layer (7),
at least one detector (11) for sensing luminescent radiation, wherein the detector (11) is arranged below the lower surface (12) of said filter (9), and
a substrate (13) that is connected with the detector (11) and comprises the electrical interface of said detector (11).

2. The waveguide sensor (3) according to claim 1, wherein the upper surface of the filter (9) is in optical contact with the lower surface of the cladding layer (7), and the lower surface of the filter (9) is in optical contact with the detector (11), and preferably the upper surface of the filter (9) contacts the lower surface of the cladding layer (7), and the lower surface of the filter (9) contacts the detector (11).

3. The waveguide sensor (3) according to claim 1, wherein the cladding layer (7) is based on an organic transparent polymer, and preferably the waveguide layer (4), the cladding layer (7) and the substrate (13) are based on an organic transparent polymer.

4. The waveguide sensor (3) according to claim 1, wherein the upper outer and/or lower inner surface of said waveguide layer (4) possess at least on recess (18) for enhancing the coupling of the light wave into said waveguide layer (4), wherein the depth of said recess (18) is preferably less than the thickness of the said waveguide layer (4) and in case that a recess is formed on the lower inner surface of said waveguide layer (4) the cladding layer (7) engages with a positive fit into said recess.

5. The waveguide sensor (3) according to claim 1, wherein the outer upper surface of said waveguide layer (4) is covered with a thin noble metal layer.

6. The waveguide sensor (3) according to claim 1, wherein the filter (9) has a high transmission for the emission radiation of fluorophores and is not translucent or poorly transmittent for the excitation radiation, and preferably the ratio of the transmission of the emission radiation over the excitation radiation of said filter is in the range of ≧10:1 to 1.000.000:1.

7. An evanescent field induced sensor system (1) that comprises a housing (2) and an integrated waveguide sensor (3) according to claim 1, wherein between the upper surface of the waveguide layer (4) and along at least a lower surface section of the housing (2) a channel (14) is formed for receiving a fluidic probe; and the luminescent radiation is generated by luminescence of a target substance as a result of their excitation by the evanescent field.

8. The system (1) according to claim 7, wherein the housing (2) comprises a laser (15), a beam shaper lens (16) and/or a prism (17).

9. The system (1) according to claim 7, wherein the housing (2) is removable connected with the integrated waveguide sensor (3).

10. The system (1) according to claim 7, wherein the housing (2) is an integral part of an medical apparatus, read out device, diagnostic apparatus or surgical instrument.

11. Use of an evanescent field based waveguide sensor (3) according to claim 1 for:
chemical or biological analysis, comprising analysis of biological fluids such as egg yolk, blood, serum or plasma;
environmental analysis, comprising analysis of water, dissolved soil extracts and dissolved plant extracts;
reaction solutions, dispersions and/or formulations analysis, comprising analysis in chemical production, in particular dye solutions or reaction solutions; and/or
quality safeguarding analysis.
